(19) United States
(12) Patent Application Publication
(10) Pub. No.: US 2005/0153353 A1
(43) Pub. Date: Jul. 14, 2005

(54) Real-time polymerase chain reaction-based genotyping assay for beta2-adrenergic receptor single nucleotide polymorphism

(76) Inventors: Bernd Meibohm, Memphis, TN (US); Manish Gupta, Memphis, TN (US); Charles R. Yates, Collierville, TN (US)

Correspondence Address: Benjamin Aaron Adler;ADLER & ASSOCIATES, 8011 Candle Lane, Houston, TX, 77071, US

(21) Appl. No.: 11/032,541

(22) Filed: Jan. 10, 2005

Related U.S. Application Data
(60) Provisional application No. 60/535,242, filed on Jan. 9, 2004.

(51) Int. Cl. C12Q 1/68; C07H 21/04

(52) U.S. Cl. 435/6; 536/24.3

(57) ABSTRACT

The present invention provides fluorescence-based real-time PCR assays for the rapid detection of β₂-adrenergic receptor single nucleotide polymorphisms (SNPs). The genotyping assay can be used to detect SNPs of human β₂-adrenergic receptor (β₂-AR) single nucleotide polymorphisms A46G and C79G.

### CROSS REFERENCE TO RELATED APPLICATION

This non-provisional application claims benefit of provisional application U.S. Ser. No. 60/535,242 filed on Jan. 9, 2004, now abandoned.

### BACKGROUND OF THE INVENTION

1. Field of the Invention

The present invention relates generally to the field of single nucleotide polymorphism genotyping. More specifically, the present invention provides a real-time polymerase chain reaction-based genotyping assay for the detection of β₂-adrenergic receptor single nucleotide polymorphisms.

2. Description of the Related Art

Chronic obstructive pulmonary disease (COPD) is characterized by decreased expiratory flow rates, increased pulmonary resistance and hyperinflation of the lung. It is a major medical problem and a leading cause of morbidity and mortality among the adult population. In the U.S., chronic obstructive pulmonary disease affects >16 million people, accounts for 13% of hospitalizations and is the fourth leading cause of death. Although cigarette smoking is the major risk factor, only 10-20% smokers develop symptomatic chronic obstructive pulmonary disease. Numerous epidemiological studies provide compelling evidence that genetic factors influence the development of chronic obstructive pulmonary disease. These factors may include the inherited deficiency of α₁-antitrypsin in certain individuals, which is relatively uncommon and explains only a very small proportion (<1%) of chronic obstructive pulmonary disease cases (Poller, W et al., 1990).

The association between chronic obstructive pulmonary disease pathogenesis and polymorphisms in several genes suspected in its pathogenesis has been studied. These include α₁-antichymotrypsin (Sandford A. J. et al., 1998; Ishii T. et al., 2000; Poller W. et al., 1993; Benetazzo M. G. et al., 1999), microsomal epoxide hydrolase (Cheng S. L. et al., 2004; Yim J. J. et al., 2000; Yoshikawa M. et al., 2000; Smith C. A. and Harrison D. J., 1997; Rodriguez F, et al., 2002), vitamin D-binding protein (Laufs J., et al., 2004; Schellenberg D. et al., 1998), glutathione-S-transferase (Yim J. J. et al., 2002; Ishii T et al., 1999; Harrison D. J. et al., 1997), cytochrome P450 1A1 (Dialyna I. A. et al., 2003), immunoglobulin-A (Ruse C. E. et al., 2003; van der Pouw Kraan T. C. et al., 2002), matrix metalloproteinases (Joos L. et al., 2002; Minematsu N. et al., 2001) and tumor-necrosis factor-α (Higham M. A. et al., 2000; Ishii T. et al., 2000; Keatings V. M. et al., 2000; Teramoto S. and Ishii T., 2001; Sakao S. et al., 2001). Results of some of these studies have been inconsistent and in most cases these polymorphisms were found not to be associated with the frequency and severity of chronic obstructive pulmonary disease. It is likely that multiple genes are operating in chronic obstructive pulmonary disease and genetic susceptibility may depend on the coincidence of several gene polymorphisms acting together. The search and identification of genetic factors that contribute to the progression of chronic obstructive pulmonary disease will clarify and deepen the understanding of the pathogenesis of chronic obstructive pulmonary disease and lead to a more specific and successful treatment.

The human β₂-adrenergic receptor (β₂-AR) is a member of the seven-transmembrane family of receptors. It is expressed in many cell types such as airway smooth muscle cells, neutrophils, eosinophils, alveolar macrophages and airway epithelial cells. The-β₂-adrenergic receptor (β₂-AR) has been identified as playing a central role in the pathogenesis, progression and/or pharmacotherapy of numerous respiratory and cardiovascular diseases including asthma, chronic obstructive pulmonary disease and hypertension. Hence, the β₂-AR agonists are used as bronchodilator drugs in the treatment of asthma and chronic obstructive pulmonary disease. These agonists are classified by their selectivity, affinity for the receptor, potency and pharmacological efficacy. The gene encoding for this G-protein coupled receptor is located on chromosome 5 (5q31-33). Thirteen single nucleotide polymorphisms occurring in this intronless gene have been identified (Drysdale C. M. et al., 2000). Five of these polymorphisms are degenerative, but four are single-point mutations resulting in single amino acid substitution in the β₂-adrenergic receptor.

Polymorphisms of the β₂-adrenergic receptor gene at nucleotide position 46 (A→G), at position 79 (C→G), and at position 491 (C→T) produce changes in the amino acid sequence at position 16 (Arg→Gly), at position 27 (Gln→Glu), and at position 164 (Thr→Ile), respectively. These changes in amino acid sequence are suggested to modulate the behavior of the β₂-receptor, alter ligand binding and the characteristics of down-regulation following agonist exposure (Ligget S. B., 2000; Joos L., 2001; Liggett S. B., 2002). Clinically, the single nucleotide polymorphisms A46G and C79G have been studied 1 5 extensively because of their high prevalence in the population and have been a focus of interest in attempts to elucidate the genetic basis of asthma, chronic obstructive pulmonary disease, hypertension, obesity, type II diabetes and cystic fibrosis (Romaino S. M. et al., 2004; Taylor D. R. and Kennedy M. A., 2001; Wang Z. et al., 2001; Taylor D. R. and Kennedy M. A., 2002). Both in vitro and in vivo evidence suggests that these two polymorphisms are functionally relevant.

Genetic variation in the β₂-adrenergic receptor might be clinically important as a determining factor affecting therapeutic responses to β₂-agonist drugs. The frequencies of polymorphisms at these positions differ between Whites, Blacks and Asian population. Both mutations have a relative high allelic frequency in normal, healthy individuals of 54% and 35% in Whites and 51% and 21% in African Americans for nucleotide positions 46 and 79, respectively (Xie et al., 1999).

One study investigated the clinical significance of the β₂-AR polymorphisms at positions 16, 27 and 164 in 65 Chinese chronic obstructive pulmonary disease patients and 41 corresponding healthy control subjects by correlating the incidence of the polymorphisms with the pulmonary function in this Chinese population (Ho, L. I. et al., 2001). This study reported that the β₂-AR polymorphism at amino acid position 16 was less prevalent in chronic obstructive pulmonary disease patients than in healthy individuals (p=0.01). Additionally, patients that were homozygous wild type for the β₂-AR polymorphism at amino acid position 27 had a higher percentage of low FEV1 percent predicted than patients that were homozygous mutant or heterozygous. However, there is considerable ethnic difference in the frequency of β₂-AR polymorphisms. Another study reported a marked difference in distribution of the functionally significant β₂-AR polymorphisms at amino acid positions 16 and 27 among African-American, Whites and Chinese individuals (Xie, H. G. et al., 1999). Such interethnic differences may be an important determinant of disease prevalence, severity and response to treatment. Thus, the results of the first study that reported the frequency and severity of chronic obstructive pulmonary disease in Chinese patients relative to β₂-AR polymorphisms cannot be extrapolated to Whites and Blacks. A subgroup of the Lung health study reported that heterozygosity for β₂-AR polymorphism at amino acid position 27 might be protective against accelerated rate of decline in lung function. However, the study population of this analysis was limited to Caucasian smokers (Joos, L et al., 2003). Since no study so far has investigated the allelic and genotype frequencies for β₂-AR polymorphisms in Caucasian and African-American chronic obstructive pulmonary disease patients, it is necessary to investigate the role of β₂-AR polymorphisms at amino acid positions 16 and 27 on the prevalence and severity of chronic obstructive pulmonary disease. This is important due to the difference in the genotype frequencies of β₂-AR polymorphisms between Whites and Blacks, which might affect the prevalence and severity of chronic obstructive pulmonary disease between the two groups.

Real-time polymerase chain reaction (PCR) has recently evolved as a fast and inexpensive methodology for allelic discrimination assays. To facilitate clarification of the association of single nucleotide polymorphisms (SNPs) in the candidate β₂- adrenergic receptor gene with the susceptibility to and progression of asthma, chronic obstructive pulmonary disease as well as cardiovascular diseases, a rapid and robust real-time PCR-based genotyping assay to detect the single nucleotide polymorphisms of β₂-adrenergic receptor at nucleotide position 46 (A→G) and 79 (C→G) would be highly desirable.

The prior art is deficient in a rapid and robust real-time PCR-based genotyping assay to detect the single nucleotide polymorphisms of the β₂-adrenergic receptor. The present invention fulfills this longstanding need and desire in the art.

### SUMMARY OF THE INVENTION

Allelic discrimination assays for genetic variants of β₂-AR previously described involved multiple steps, were time-consuming and relatively expensive for large-scale clinical and epidemiological studies (Gray M. R., 1992; Newton C. R. et al., 1989; Kim S. et al., 2003; Yoshida N. et al., 2002). The present invention provides a rapid, inexpensive and robust real-time polymerase chain reaction (PCR)-based method to detect single nucleotide polymorphisms of the β₂-adrenergic receptor. Discrimination between wild type and mutant alleles was achieved using PCR amplification of specific alleles modified to prevent non-Watson Crick base pairing (Okimoto & Dodgson, 1996; Sommer et al., 1992; Bottema et al., 1993; Newton et al., 1989). Two key nucleotide mismatches are required for allelic discrimination. The first nucleotide difference between primers used to discriminate between wild type and mutant alleles was located at the 3′ terminal base. However, a single base pair difference at the 3′ end of the primer is insufficient, in most cases, to achieve allelic discrimination. An additional internal nucleotide mismatch (typically within 5 base pairs of the 3′ end) is required for specific amplification of either the wild-type or mutant allele. Thus, a second nucleotide mismatch located two to three bases from the 3′ end for both the wild-type and mutant-specific primers was included to generate an internal primer/template mismatch that prevents amplification of the nonmatching primer. This assay provides significant advantages over current genotyping techniques by using SYBR Green I fluorescent dye for real-time detection and melting curve analysis of PCR product.

The present invention provides a genotyping assay to detect β₂-AR (β₂-adrenergic receptor gene) single nucleotide polymorphism (SNP) A46G. A46G is suggested to result in altered receptor function after agonist exposure. PCR reactions for genotyping A46G using allele-specific primers were conducted in separate tubes. PCR amplification was monitored by Smart Cycler (Cepheid, Sunnyvale, Calif.) using SYBR™ Green I, a non-specific double stranded DNA intercalating fluorescent dye. PCR growth curves exceeding the threshold cycle were considered positive. Fluorescence melt-curve analysis was used to corroborate results from PCR growth curves.

Using PCR growth curves, the assay disclosed herein accurately determined hetero- and homozygosity for A46G. Genotype assignments based on PCR growth curve, melt-curve analysis, agarose gel electrophoresis, and direct DNA sequencing results of PCR products were in perfect agreement which demonstrated the reliability and discriminating power of this technique. Although, this technique has been used previously to genotype genetic variants of other genes (Yates C. R. et al., 2003; Song, P. et al., 2002; Gupta M. et al., 2004), the present invention demonstrates the usefulness of this assay to evaluate patients identified from the baseline ‘Health, Aging and Body Composition’ study, with or without airflow limitation according to the criteria of the American Thoracic Society, to investigate the association between airflow limitation and β₂-AR polymorphisms. Thus, the present invention provides a rapid β₂-adrenergic receptor genotyping method that can be used by a person having ordinary skill in this art to assess the contribution of β₂- adrenergic receptor single nucleotide polymorphisms to the prevalence and severity of chronic obstructive pulmonary disease.

In one embodiment of the present invention, there are primer pairs consisting of an allele specific sense primer and a reverse primer for genotyping A46G single nucleotide polymorphism of human β₂-adrenergic receptor (β₂₋AR) gene.

In another embodiment of the present invention, there are primer pairs consisting of an allele specific sense primer and a reverse primer for genotyping C79G single nucleotide polymorphism of human β₂-adrenergic receptor (β₂₋AR) gene.

In yet another embodiment of the present invention, there is a method of genotyping A46G single nucleotide polymorphism of human β₂-adrenergic receptor (β₂₋AR) gene. This method comprises the step of extracting DNA from sample of an individual. The DNA is then amplified in separate PCR reactions comprising wild type allele specific sense primer and a reverse primer or mutant allele specific sense primer and the reverse primer followed by identification of the products of the DNA amplification. The presence of products amplified by the wild type allele specific sense primer and the reverse primer and the mutant allele specific sense primer and the reverse primer indicate that the individual has the A46G single nucleotide polymorphism of human β₂-adrenergic receptor (β₂₋AR) gene.

In further yet another embodiment of the present invention, there is a method of genotyping C79G single nucleotide polymorphism of human β₂-adrenergic receptor (β₂₋AR) gene. This method comprises the same method steps as described earlier.

In still yet another embodiment of the present invention, there is a method of genotyping A46G single nucleotide polymorphism of human β₂-adrenergic receptor (β₂₋AR) gene. This method comprises the same method steps as discussed earlier using primer pairs having SEQ ID No. 1 and SEQ ID No. 3 or SEQ ID No. 2 and SEQ ID No. 3.

In another embodiment of the present invention, there is a method of genotyping C79G single nucleotide polymorphism of human β₂-adrenergic receptor (β₂₋AR) gene. This method comprises the same method steps as discussed earlier using primer pairs having SEQ ID No. 4 and SEQ ID No. 6 or SEQ ID No. 5 and SEQ ID No. 6.

In still another embodiment of the present invention, there are kits to genotype A46G and C79G single nucleotide polymorphism of human β₂-adrenergic receptor (β₂₋AR) gene. This kit comprises: (a) Allele specific sense primers, and (b) Reverse primers.

In still yet another embodiment of the present invention, there is a method of evaluating clinical significance of A46G single nucleotide polymorphism of human β₂-adrenergic receptor (β₂₋AR) gene in individuals with disease. In addition to the method steps discussed earlier, this method also comprises the step of comparing the incidence of the polymorphism in said individuals to the incidence of the polymorphism in samples from control individuals who do not have the disease, where increased incidence of the polymorphism in individuals with the disease compared to the control individuals indicates that the polymorphism is clinically significant in the disease.

In further yet another embodiment of the present invention, there is a method of evaluating clinical significance of C79G single nucleotide polymorphism of human β₂-adrenergic receptor (β₂₋AR) gene in individuals with disease. This method comprises of the same steps as discussed above.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1A-1C show β₂-adrenergic receptor A46G allelic discrimination by real-time analysis using the Smart Cycler. Plot of fluorescence versus cycle number using human genomic DNA obtained from individuals with A/A (FIG. 1A), A/G (FIG. 1B), or G/G (FIG. 1C) genotypes. Interrogation for the presence of either the A or G allele was conducted in physically separate tubes using the common reverse primer 46R coupled with either the wild type specific primer 46FW or the mutant-specific primer 46FM. PCR growth curves that exceed the threshold fluorescence indicate specific PCR product formation. Cₜ: threshold cycle.

FIGS. 2A-2C show melt curve analysis of PCR products using SYBR™ Green I. Melt curves were converted to melt peaks by plotting the negative first derivative of the fluorescence versus temperature ([−dF/dt]). Plot of [−dF/dt] versus temperature obtained after amplification of A/A (FIG. 2A), A/G (FIG. 2B), G/G (FIG. 2C) genomic DNA using the common reverse primer 46R coupled with either the wild type specific primer 46FW or the mutant-specific primer 46FM. The melt temperature (Tₘ=87° C.) was identical for PCR products formed using either the wild type or mutant-specific primers.

FIG. 3 shows the identification of polymorphisms in nucleic acid 46 of the β₂-adrenergic receptor in three patients by conventional modified allele-specific PCR. An ethidium bromide-stained 2% agarose gel containing PCR fragments (204 bp) was run to confirm real-time PCR results. Odd-numbered lanes contain PCR fragments after amplification with 46R and 46FW. Even-numbered lanes contain PCR fragments after amplification with 46R and 46FM. PCR products amplified from genomic DNA with different 46 genotypes were loaded as follows: homozygotes A/A (lanes 1 and 2), heterozygotes A/G (lanes 3 and 4), and nullizygotes G/G (lanes 5 and 6). M: Molecular marker which contains a 100-bp DNA ladder.

FIG. 4 shows the identification of polymorphisms in nucleic acid 79 of the β₂-adrenergic receptor in two patients by conventional modified allele-specific PCR. An ethidium bromide-stained 2% agarose gel containing PCR fragments (158 bp) was run to confirm real-time PCR results. Odd-numbered lanes contain PCR fragments after amplification with 79R and 79FW. Even-numbered lanes contain PCR fragments after amplification with 79R and 79FM. PCR products amplified from genomic DNA with different 79 genotypes were loaded as follows: homozygotes C/C (lanes 1 and 2), heterozygotes C/G (lanes 3 and 4). No subject who has nullizygotes 79G/G was found. M: Molecular marker which contains a 100-bp DNA ladder.

FIG. 5 shows the sequence analysis for PCR products containing both the sites of polymorphisms at nucleotide position 46 and 79 of the β₂-adrenergic receptor. The sense primer for sequencing is: 5′-GCTCACCTGCCAGACTGC-3′, SEQ ID NUMBER; 7, and the antisense primer for sequencing is:

| | |
|---|---|
| 5′-CAGCAGGTCTCATTGGCATA-3′,. | SEQ ID Number: 8 |

FIGS. 6A-6B show the genotype distribution based on β₂-AR polymorphisms in subjects. FIG. 6A shows the genotype distribution for polymorphism at nucleotide position 46 (amino acid position 16) in subjects with airflow limitation (Cases) and without airflow limitation (controls). FIG. 6B shows the genotype distribution for polymorphism at nucleotide position 79 (amino acid position 27) in subjects with airflow limitation (Cases) and without airflow limitation (controls).

FIGS. 7A-7B show the genotype distribution for β₂-AR polymorphism at amino acid position 27 in Whites and Blacks. FIG. 7A shows the genotype distribution with airflow limitation (cases) and in those without airflow limitation (controls) in Whites. FIG. 7B shows genotype distribution with airflow limitation (cases) and in those without airflow limitation (controls) in Blacks.

FIG. 8 shows distribution of β₂-AR haplotypes at positions 16 and 27 in cases and controls. There were no significant differences in the haplotype frequencies between cases and controls.

FIGS. 9A-9B show distribution of β₂-AR polymorphisms stratified by FEV 1. FIG. 9A shows distribution of β₂-AR-16 polymorphisms stratified by FEV1 in the Whites and Blacks. There was no significant difference in genotype frequencies between the groups (P=0.20 for a 3 X 3 contingency table). FIG. 9B shows distribution of β₂-AR-27 polymorphisms stratified by FEV1 in the Whites and Blacks. A significant difference in genotype frequency was observed between the three groups (P=0.04 for a 3 X 3 contingency table).

FIGS. 10A-10B show distribution of the β₂-AR-27 polymorphism stratified by FEV1. FIG. 10A shows distribution of the β₂-AR-27 polymorphisms stratified by FEV1 in the Whites. A significant difference in genotype frequency was observed between the three groups (P=0.02 for a 3 X 3 contingency table). FIG. 10B shows distribution of β₂-AR-27 polymorphisms stratified by FEV1 in the Blacks. There was no significant difference in genotype frequencies between the groups (P=0.24 for a 3 X 3 contingency table).

FIG. 11 shows distribution of β₂-AR haplotypes stratified by FEV1 in the whites. Arg16/Glu27 was particularly very rare in the sample population and is not shown in the figure. Gly16/Glu27 haplotype was most frequent in subjects with FEV1 <50%, whereas Arg16/Gln27 haplotype was most common in subjects with FEV1>79%.

### DETAILED DESCRIPTION OF THE INVENTION

Fluorescence-based single nucleotide polymorphism detection assays offer several important advantages over traditional PCR approaches used to determine genotype (e.g. sequencing of PCR products and restriction fragment length polymorphism, RFLP). First, restriction fragment length polymorphism analysis can in some instances result in significant false positive rates as a result of incomplete restriction enzyme digestion or the presence of other mutations close to the mutation of interest. Second, fluorescence-based genotyping assays are more amenable to high-throughput screening, as they do not require extensive post-amplification manipulation.

Commonly used fluorescence-based PCR techniques for single nucleotide polymorphism detection include the use of either the nonspecific DNA intercalating dye SYBR™ Green I or an allele-specific fluorogenic probe (i.e. Taqman). In many instances, the use of SYBR™ Green I is more cost-effective when applied to haplotype analysis of genes with multiple allelic variants since it does not require the synthesis of numerous allele-specific fluorogenic probes.

In certain embodiments of the present invention, the use of allele-specific primers containing an additional internal mismatch obviates the need for extensive optimization of PCR amplification conditions associated with traditional PCR amplification of specific alleles.

Current methods for genotyping the β₂-adrenergic receptor include PCR amplification followed by sequencing and fluorogenic probe-based PCR assays. The simple, rapid, inexpensive, reproducible, and reliable real-time PCR genotyping methods presented here constitute a significant improvement over current techniques. Using this approach, genotyping results can be obtained within 2 hours of whole blood or tissue procurement. Importantly, these techniques described herein are also generally applicable in laboratories lacking access to real-time PCR equipment because allelic discrimination can be determined using traditional PCR and agarose gel electrophoresis. The reliability and discriminating power of this technique is also demonstrated by comparing the results of this real-time PCR-based assay with direct sequencing

Thus, the present invention is directed to primer pairs consisting of an allele specific sense primer and a reverse primer for genotyping A46G single nucleotide polymorphism of human β₂-adrenergic receptor (β₂-AR) gene. The allele specific sense primer has an additional internal nucleotide mismatch three bases from the 3′ terminus. The nucleotide sequence at the 3′ end of the wild type allele specific sense primer is at least 85% or completely homologous to SEQ ID No. 1 whereas the nucleotide sequence at the 3′ end of the mutant allele specific sense primer is at least 85% or completely homologous to SEQ ID No. 2. Specifically, the homology at 3′ end is within the first 16 base pairs. The reverse primer used in the PCR reaction has a nucleotide sequence which is 90% or completely homologous to SEQ ID No. 3.

The present invention is also directed to primer pairs consisting of an allele specific sense primer and a reverse primer for genotyping C79G single nucleotide polymorphism of human β₂-adrenergic receptor (β₂-AR) gene. The allele specific sense primers have same characteristics as discussed earlier. Additionally, the nucleotide sequence at the 3′ end of wild type allele specific sense primer is at least 85% or completely homologous to SEQ ID No. 4 whereas the nucleotide sequence at the 3′ end of mutant allele specific sense primer is at least 85% or completely homologous to SEQ ID No. 5. Specifically, the homology at the 3′ end is within the first 16 base pairs. The reverse primer used in the PCR reaction has a nucleotide sequence which is 90% or completely homologous to SEQ ID No. 6.

The present invention is further directed to a method of genotyping A46G single nucleotide polymorphism of human β₂-adrenergic receptor (β₂-AR) gene, comprising the steps of: extracting DNA from sample of an individual, amplifying the DNA in separate PCR reactions comprising wild type allele specific sense primer and a reverse primer or mutant allele specific sense primer and the reverse primer, and identifying the products of the DNA amplification, wherein the presence of products amplified by the wild type allele specific sense primer and the reverse primer and the mutant allele specific sense primer and the reverse primer indicate that the individual has the A46G single nucleotide polymorphism of human β₂-adrenergic receptor (β₂₋AR) gene. All other aspects regarding the sense and reverse primers used in the PCR reaction are as discussed earlier.

Additionally, the mismatch nucleotide in the sense primers has little effect on specific PCR product yield but drastically reduces non-specific product yield to undetectable levels. Generally, the products of DNA amplification are identified by a method selected from the group consisting of real-time fluorescence-based analysis, melt curve analysis and gel electrophoresis. Specifically, the gel electrophoresis identifies a product of 204 base pairs that corresponds to a product with A46G single nucleotide polymorphism of human β₂-adrenergic receptor (β₂₋AR) gene.

The present invention is also directed to a method of genotyping C79G single nucleotide polymorphism of human β₂-adrenergic receptor (β₂₋AR) gene, comprising the steps discussed earlier. All other aspects regarding the sense and reverse primers and the methods for identifying the products of DNA amplification are as discussed earlier. Specifically, the gel electrophoresis identifies a product of 158 base pairs that corresponds to a product with C79G single nucleotide polymorphism of human β₂-adrenergic receptor (β₂₋AR) gene.

The present invention is further yet directed to a method of genotyping A46G single nucleotide polymorphism of human β₂-adrenergic receptor (β₂₋AR) gene, comprising the steps discussed earlier using primer pairs consisting of SEQ ID No. 1 and SEQ ID No. 3 or SEQ ID No. 2 and SEQ ID No. 3. All other aspects regarding the sense primers, method for identifying the product of DNA amplification and the size of product identified by gel electrophoresis is discussed earlier.

Additionally, the present invention is also directed to a method of genotyping C79G single nucleotide polymorphism of human β₂-adrenergic receptor (β₂₋AR) gene, comprising the steps discussed earlier using primer pairs consisting of SEQ ID No. 4 and SEQ ID No. 6 or SEQ ID No. 5 and SEQ ID No. 6. All other aspects regarding the sense primers, the methods of identifying product of DNA amplification and the size of product identified by gel electrophoresis is discussed earlier.

The present invention is further yet directed to kits for genotyping A46G and C79G single nucleotide polymorphisms of human β₂-adrenergic receptor (β₂₋AR) gene. These kits comprise: (a) Allele specific sense primers, and (b) Reverse primers. All other aspects regarding these primers for the specific polymorphism are as discussed earlier.

The present invention is also directed to a method of evaluating clinical significance of A46G single nucleotide polymorphism of β₂-adrenergic receptor (β₂₋AR) gene in individuals with disease, comprising: extracting DNA from samples of the individuals, genotyping said DNA for the A46G polymorphism, and comparing the incidence of the polymorphism in the individuals to the incidence of the polymorphism in samples from control individuals who do not have the disease, wherein increased incidence of the polymorphism in individuals with the disease compared to the control individuals indicates that the polymorphism is clinically significant in that disease. The disease is a respiratory or cardiovascular disease including asthma, chronic obstructive pulmonary disorder or hypertension. All other aspects regarding the method of genotyping, sense and reverse primers are as discussed earlier. Since the significance of a polymorphism may differ based on the ethnicity, this method further comprises: correlating the incidence of the polymorphism with the disease in the population of individuals of same ethnicity to determine the prevalence, severity and the response to treatment in the population.

The present invention is further directed to a method evaluating clinical significance of C79G single nucleotide polymorphism of β₂-adrenergic receptor (β₂₋AR) gene in individuals with disease, comprising the same steps as discussed above. Additionally, all aspects regarding the types of disease, method of genotyping, sense and reverse primers and determination of prevalence, severity and the response to treatment in the population are as discussed earlier.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the DNA and methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### EXAMPLE 1

### β₂-AR Single Nucleotide Polymorphisms Genotyping

The present example describes real-time PCR assays for the rapid detection of the β₂-adrenergic receptor single nucleotide polymorphisms A46G and C79G. These methods can be readily applied to investigate the effect of the β₂-adrenergic receptor polymorphic expression on pathogenesis, progression and/or pharmacotherapy of numerous respiratory and cardiovascular diseases including asthma, chronic obstructive pulmonary disease (COPD), and hypertension.

### EXAMPLE 2

### Primer Design

Discrimination between wild type and mutant alleles was achieved using PCR amplification of specific alleles modified to prevent non-Watson Crick base pairing (Okimoto & Dodgson, 1996; Sommer et al., 1992; Bottema et al., 1993; Newton et al., 1989). Since Taq DNA polymerase lacks 3′ to 5′ exonuclease activity, a primer with a mismatch in the 3′ terminal region with regard to the template will be amplified with reduced efficiency, allowing discrimination between matched and mismatched templates. Two forward primers and a common reverse primer were designed based on the nucleotide difference at the 3′ terminal base based upon the published β₂-AR sequence (Sequence Accession No. M15169) using Primer3 program (Whitehead Institute for Biomedical Research, http://www. Genome. Wi.mit.edu/cgi-bin/primer/primer3_www.cgi).

Briefly, the allelic discrimination for A46G was achieved by designing two sense primers (46FW and 46FM) based on the nucleotide difference (A or G) located at the 3′ terminal base. An additional nucleotide mismatch (A to T) located 2 or 3 bases from the 3′ termini of the allele specific primers (46FW and 46FM) was incorporated (Table 1). These changes were made to improve the amplification specificity and to prevent the generation of non-specific products, which could otherwise occur by the annealing and extension of the 46FW primer to non-specific template.

A similar strategy was used to achieve allelic discrimination for C79G (Table 1). Oligo Toolkit™ (www.operon.com) was used to detect hairpin structures and primer-dimers. Primers were synthesized by Integrated DNA Technologies, Inc. (Coralville, Iowa). Expected amplicon lengths were 204 base pairs and 158 base pairs for A46G and C79G, respectively.

### EXAMPLE 3

### Real-Time PCR Amplification

Genomic DNA was obtained for 10 Blacks and 10 Northern Europeans from the Human Genetic Cell Repository, sponsored by the National Institute of General Medical Sciences (http://locus.umdnj.edu/nigms). The use of Human Genetic Cell Repository samples was approved by the University of Tennessee Institutional Review Board. Polymorphisms were detected by PCR amplification of specific alleles (PASA) (Newton C. R. et al., 1989; Song P. et al., 2002; Gupta M. et al., 2004; Bottema C. D. et al., 1993; Okimoto R. et al., 1996) on a SmartCycler™ (Cepheid, Sunnyvale, Calif.) using SYBR™ Green I (Molecular Probes, Eugene, Oreg.), a nonspecific double-stranded DNA intercalating fluorescent dye.

Thus, to achieve allelic discrimination between wild type and mutant alleles, two physically separate PCR reactions containing either wild type specific primer (46FW) or mutant-specific primer (46FM) and a common primer (46R) was performed. All reactions were carried out in a total volume of 25 μL. Reaction conditions were identical for A46G and C79G except where noted. Each reaction mixture contained a 1:12,500 dilution of SYBR™ Green I nucleic acid gel stain 10,000× in dimethyl sulfoxide (DMSO) (Molecular Probes); 0.2 mM of dATP, dCTP, dGTP, and dTTP mixture; 200 nM of both forward and reverse primers; 1.0 U of Taq DNA polymerase (Promega, Madison, Wis.); 6% DMSO; 1× SmartCycler™ additive reagent (a 5× additive reagent containing bovine serum albumin at 1 mg/mL, Trehalose at 750 nM, and Tween-20 at 1% v/v ) (Cepheid, Sunnyvale, Calif.), and 10 ng of genomic DNA in 1×PCR buffer (pH 8.3, 10× solution containing 100 mM Tris-HCl, 500 mM KCl, 15 mM MgCl₂ and 0.01% gelatin) (Sigma, St. Louis, Mo.).

The amplification program for both A46G and C79G consisted of initial denaturation of 95° C. (5 minutes) followed by 27 cycles of 95° C. (15 seconds), annealing at 60° C. (45 seconds), extension at 72° C. (30 seconds). After amplification, melt analysis was performed by heating the reaction mixture from 60° C. to 95° C. at the rate of 0.2° C./second. A negative control without DNA template was run with every assay to demonstrate the overall specificity. PCR products containing both the sites of polymorphism at nucleotide position 46 and 79 were generated for sequencing using the sense primer (5′-GCTCACCTGCCAGACTGC-3′, SEQ ID Number: 7) and the antisense primer (5′-CAGCAGGTCTCATTGGCATA-3′, SEQ ID Number: 8). PCR products were isolated by QlAquick (Qiagen, Valencia, Calif.) and analyzed using direct sequencing (ABI Prism® 3100, Applied Biosystems, Foster City, Calif.).

### EXAMPLE 4

### PCR Product Analysis

The real-time fluorescence signal generated by the nonspecific double-stranded DNA binding dye SYBR™ Green I was analyzed using the SmartCycler™ software. A threshold cycle (Cₜ) was determined for each sample using the exponential growth phase and the baseline signal from fluorescence versus cycle number plots. A sample was deemed positive if fluorescence exceeded the threshold. Threshold fluorescence level was automatically set by the SmartCycler™ software. Melting curve analysis was performed by slowly heating DNA fragments in the presence of the dsDNA-specific fluorescent dye SYBR™ Green I. As the sample is heated, fluorescence (F) rapidly decreases when the melting temperature of a particular fragment is reached. Negative first derivative peaks ([−dF/dT] vs. temperature), which are characteristic of the PCR product melt temperature, were used to identify specific PCR products. Genotypes were assigned based on the threshold cycle Cₜ and the presence of a specific melting curve peak. Amplification reactions were also routinely checked for the presence of nonspecific products by agarose gel electrophoresis.

### EXAMPLE 5

### β₂-AR Genotyping Results

Allele-specific primers containing an additional nucleotide mismatch 2 or 3 bases from their 3′ termini had little effect on specific PCR product yield. However, nonspecific PCR product yield was drastically reduced to basically undetectable levels. Consequently, PCR conditions were optimized such that the threshold cycle (Cₜ) was exceeded only when specific amplification occurred (i.e., only in the presence of a primer: template match).

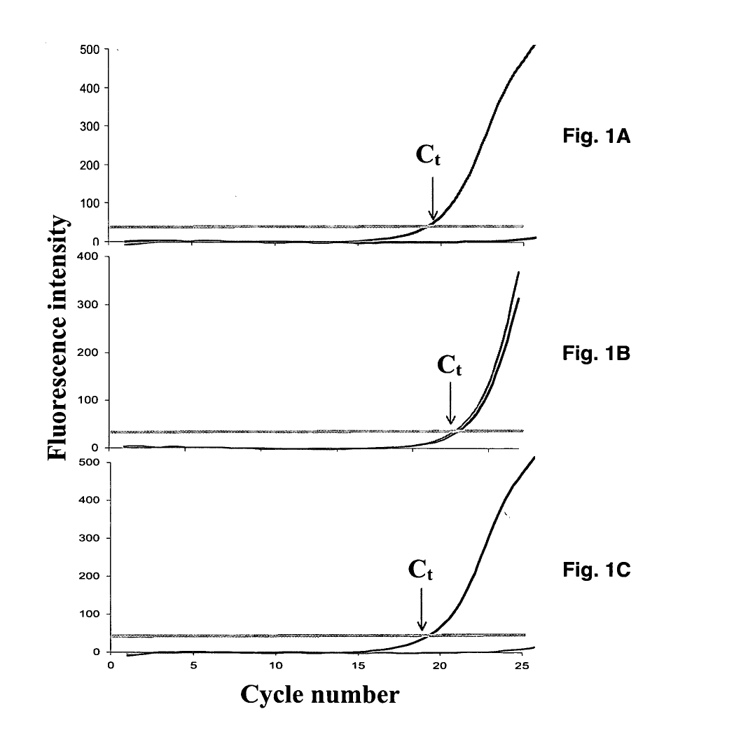

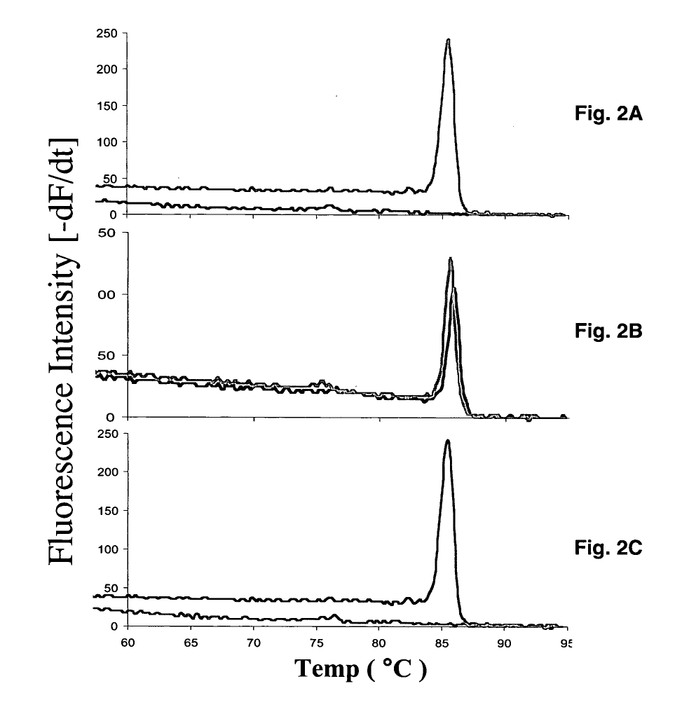

When primers 46R and 46FW were used to amplify homozygous 46A genomic DNA, the PCR growth curve exceeded the Cₜ value at approximately 21 cycles (FIG. 1A), and the melt analysis (negative first derivative) yielded a characteristic sharp peak at approximately 87° C. for the product (FIG. 2A).

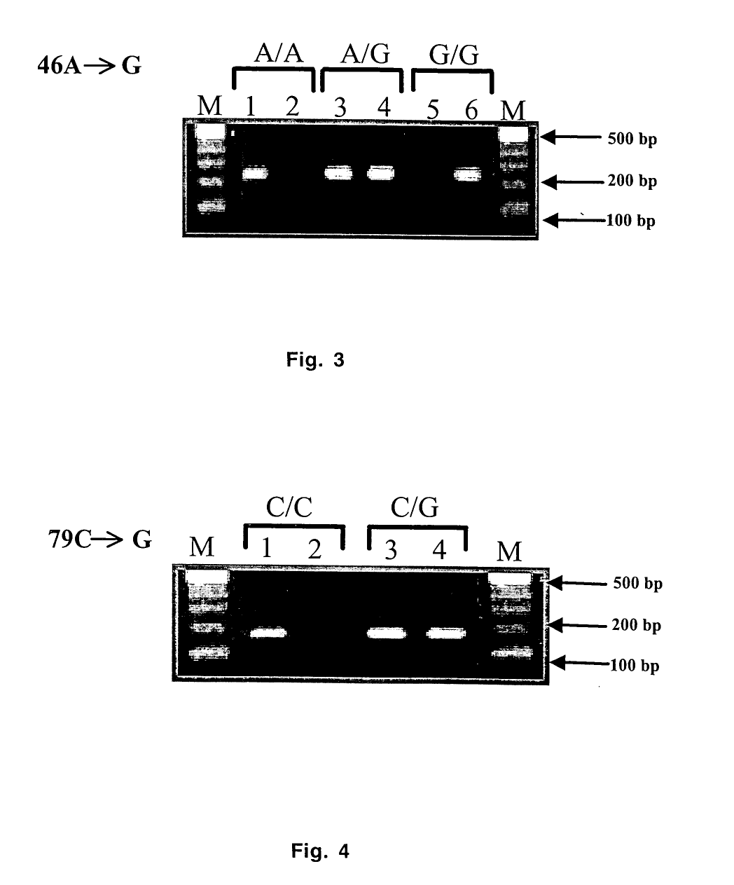

However, PCR growth curves remained at approximately background fluorescence, and no distinct melt analysis peak was noted when primers 46R and 46FM were used to amplify homozygous 46A genomic DNA (FIGS. 1A and 2A). Accordingly, agarose gel electrophoresis yielded the expected 204-bp fragment when homozygous 46A DNA was amplified with primers 46R and 46FW but no bands were visualized after homozygous 46A DNA was amplified using primers 46R and 46FM (FIG. 3). In contrast, amplification of homozygous 46G DNA using primers 46R, 46FM, and 46FW produced specific products only when primers 46R and 46FM were used (FIGS. 1C and 2C and 3).

Overlapping PCR growth curves yielding similar Cₜ values were obtained when genomic DNA heterozygous for A46G was amplified using wild-type and mutant-specific primers (FIG. 1B). In addition, a distinct melt analysis peak was present after amplification with both wild-type and mutant-specific primers (FIG. 2B). Results from real-time PCR corroborate conventional PCR results (FIG. 3) and accurately predict the presence of both wild-type and mutant 46 alleles in the heterozygote control.

Results obtained from optimization and application of the C79G genotyping assay to individuals with CC, CG genotypes were similar to those reported for A46G (data not shown). Melt analysis yielded a characteristic sharp peak at approximately 86° C. (data not shown). Results from real-time PCR corroborate conventional PCR results (FIG. 4) and accurately predict the presence of both wild type and mutant 79 alleles in the heterozygote control.

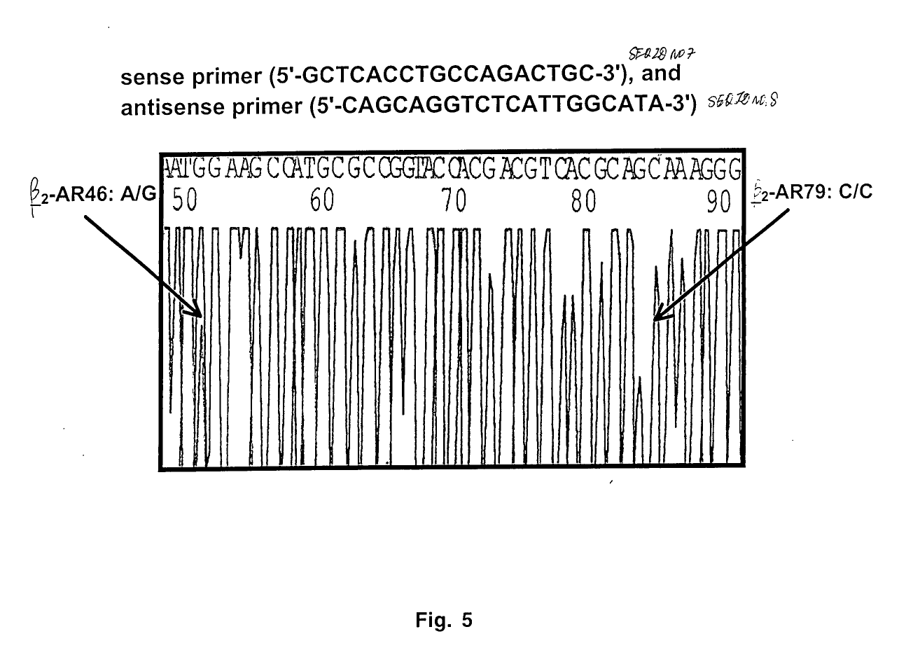

The exponential growth phase and melt peak of the specific PCR product was monitored for a set of positive controls. For the homozygous wild-type subjects, a melt peak was observed only with the wild-type primers (Table 1) at ˜87° C. and ˜86° C. for46A and 79C, respectively. For homozygous mutant subjects, a melt peak was observed with only mutant-specific primers (Table 1) at ˜87° C. and ˜86° C. for 46G and 79G, respectively. For individuals heterozygous for A46G and C79G, a melt peak was observed at ˜87° C. and ˜86° C. respectively after PCR amplification with wild type and mutant specific primers. These results were further confirmed with direct sequencing (FIG. 5).

The validity of the present methods was verified by testing 20 individuals (10 Northern Europeans and 10 African Americans) comprising homozygotes (A/A), heterozygotes (A/G) and nullizygotes (G/G) for A46G, and homozygotes (C/C) and heterozygotes (C/G) for C79G. The validity for individuals with a nullizygote (G/G) mutation for C79G was also tested using another set of samples. The genotype distribution was in Hardy-Weinberg equilibrium.

Therefore, a rapid, inexpensive and robust PCR-based screening methodology for β₂-adrenergic receptor genotyping which is a significant improvement over current genotyping techniques with regard to time and starting amount of genomic DNA was developed and validated. Allele specific amplification can also be performed with traditional thermal cyclers followed by agarose gel electrophoresis. This genotyping method can be used to assess the allelic frequency of 46G and 79G in individuals and determine their clinical significance in different populations with various disease states.

TABLE 1
Primer Sequences For β₂-AR Genotyping

| Sequence | | Annealing | | | Product |
|---|---|---|---|---|---|
| Accession No. | Polymorphism | Position | Primer | Sequence | size, bp |
| β₂-AR | 46 A/G | 1614-1633 | 46FW- | 5′ CTTCTTGCTGGCACCCATTA 3′ | 204 |
| M15169 | | | | (SEQ ID NO: 1) | |
| | | | 46FM- | 5′ CTTCTTGCTGGCACCCATTG 3′ | |
| | | | | (SEQ ID NO: 2) | |
| | | 1817-1798 | 46R- | 5′ CAGGCCAGTGAAGTGATGAA 3′ | |
| | | | | (SEQ ID NO: 3) | |
| | 79 C/G | 1647-1666 | 79FW- | 5′ GGACCACGACGTCACGCAAC 3′ | 158 |
| | | | | (SEQ ID NO: 4) | |
| | | | 79FM- | 5′ GGACCACGACGTCACGCAAG 3′ | |
| | | | | (SEQ ID NO: 5) | |
| | | 1804-1785 | 79R- | 5′ TGATGAAGTAGTTGGTGACC 3′ | |
| | | | | (SEQ ID NO:6) | |

FW and FM indicate forward primers for wild type and mutant allele, respectively. R indicates reverse primer. Nucleotides in underline indicate the site of polymorphism and corresponding wild type and mutant nucleotides. Nucleotides in italics indicate nucleotide mismatches 2 or 3 bases from the 3′ termini of the published sequence, which had little effect on specific PCR product yield but non-specific PCR product yield was drastically reduced to undetectable levels.
Discussion

Genotyping of β₂-adrenergic receptor variants should provide valuable information for future studies in medical sciences as well as in human genetics. Most of the current methods for β₂-adrenergic receptor genotyping require post-PCR handling, such as agarose or microtitre array diagonal gel electrophoresis, and thus are time-consuming and may increase the risk of PCR contamination (Xie H. G. et al., 1999; Gray M. R. 1992). Newer methods avoid these limitations but use allele-specific fluorescent probes, which are relatively expensive (Lucas T. et al., 2004). To solve this problem, refined methods are required for rapid and accurate genotyping of the β₂-adrenergic receptor gene.

Real-time PCR is an innovative way for detection and quantification of PCR amplification fragments. In the present study, a rapid, inexpensive and robust PCR-based screening methodology for β₂-adrenergic receptor genotyping, which is a significant improvement over current genotyping techniques with regard to time and starting amount of genomic DNA was developed and validated. This method takes advantage of the fluorescent property of SYBR™ Green I dye and the melting curve analysis that allow the detection and distinction of different length of PCR products. The genotyping method used here offers several advantages over traditional PCR approaches used to determine genotype (e.g., sequencing of PCR products and restriction fragment length polymorphism [RFLP]). Also, the use of SYBR™ Green I was more cost-effective compared to other fluorescence-based PCR techniques for SNP detection.

In addition, the use of allele specific primers containing an additional mismatch eliminates the need for extensive optimization of PCR conditions, which reduces time and effort during assay setup and increases assay robustness during sample analysis. Hence, this method is more amenable to high-throughput screening, due to it is high sensitivity, high specificity, relatively low cost, rapid performance and lack of extensive post amplification manipulation.

Allele specific amplification can also be performed with traditional thermal cyclers followed by agarose gel electrophoresis, thus making this technique universally applicable. This genotyping methodology has been tested and validated for achieving high throughput on ABI Prism™ 7000 Sequence Detection System using a 96-well format with the exactly same conditions as the SmartCycler™ (Cepheid, Sunnyvale, Calif.). The genotype of every specimen at nucleotide positions 46 and 79 was revealed correctly by this real-time PCR assay, indicating the high specificity of this technique.

With no need for post-PCR handling and increased sensitivity using fluorescence chemistry, PCR cycles were reduced to 27 cycles. Less than one hour was needed to complete the setting and performance of 27 PCR cycles, and data analysis. Additionally a decreased amount of genetic starting material is required for this PCR assay compared to the traditional genotyping approaches like restriction fragment length polymorphism (RFLP) (Paschke T. et al., 2001; Allen R. A. et al., 2001). The method is extremely accurate, robust, and can be optimized in a simple and predictable manner. By virtue of its simplicity, the method is versatile and cost-effective with potential for use in industrial scale genetic studies or in the clinical diagnostic setting. This genotyping method will be used to assess the allelic frequency of these alleles and determine their clinical significance in different populations with various disease states. One such use of this method is discussed in the following examples.

### EXAMPLE 6

### Study Population

In order to investigate the racial differences in the association of β₂-AR polymorphisms with prevalence and severity of COPD, two hundred and sixty-two subjects (Whites=147, Blacks=115) with airflow limitation (out of 2223 subjects with spirometry data usable for analysis) were selected from the Health, Aging and Body Composition (Health ABC) study. The design of this cohort study has been described more extensively previously (Waterer, G. W. et al., 2001). Study participants were well-functioning subjects aged 70 to 79 in the Memphis, Tenn. and the Pittsburgh, Pa. vicinities. These individuals were matched by gender, race and smoking status with 524 subjects of Health ABC with no airflow limitation. Thus, the number of controls was twice the number of individuals with airflow limitation identified in Health ABC at baseline. A biased random sample was obtained for the control group to ensure that the control group contains a similar number of current or former smokers with similar smoking history compared to the COPD group. This allowed analyzing for a gene-environment (smoking) interaction.

Demographics, smoking status and history, respiratory symptoms, medical diagnosis of respiratory disease, medication and pulmonary function test data was obtained from Health ABC database for these participants. The lung function was assessed by FEV1 % predicted, that is FEV1 adjusted for age, height and sex. The individuals with airflow limitation were further divided into the following three groups by their FEV1 values according to the GOLD staging system for severity of the COPD (Dialyna et al., 2003): FEV1<50% of predicted (moderate IIB and severe III); FEV1 50 to 79% of predicted (moderate IIA); and FEV1>79% of predicted (mild I).

Additionally, spirometry assessments and other measures of functional capacity such as PPPEF, PPFEV6, PPFVC, FEV1 from the best curve with repetitive measures, FEV1/FVC, ease walking quarter of a mile (EASEQM), ease of walking a mile (EASE1M), walking speed (m/sec) over 2 minute (TWOMINSD), walking speed (m/sec) over 400 meters (MTR400SD), Body Mass Index (BMI) and EPESE performance battery score (EPESEPPB) from the Health ABC study subjects was obtained. Spirometry was conducted at the Year 1 and Year 5 exams. EASEQM, EASE1M, BMI were monitored at Year 1-6 whereas TWOMINSD and MTR400SD were estimated at Year 1, Year 2, Year 4 and Year 6.

### EXAMPLE 7

### Genotyping Methods

Genotyping was performed using an allelic discrimination assay with PCR amplification of specific alleles and subsequently confirmed by agarose gel electrophoresis and direct sequencing as described earlier.

### EXAMPLE 8

### Statistical Analysis

The frequency distribution of β₂-AR polymorphic genotypes by case-control status was examined by chi-square tests. The association of the polymorphism genotype parameters and the pulmonary function test between healthy subjects and individuals with airflow limitation was examined by the chi-square test for trend in binomial proportions. Deviations from Hardy-Weinberg equilibrium (HWE) were tested by a χ² test of observed and expected cell counts with the degrees of freedom (df) adjusted by the number of independent frequencies estimated. To adjust for additional covariates, the data were analyzed by multiple logistic regression with airflow limitation as the outcome variable. For all analyses the significance level was set at α=0.05. For an identified association, the odds ratio (OR) (and its 95% confidence interval [95% CI]) was computed as an approximation of the relative risk. The statistical analyses were done using the SAS 9.0 and SPLUS 6.0 package.

In Health ABC Study, 262 individuals were identified at baseline as having airflow limitation based spirometry testing. Airflow was analyzed as a continuum from normal to severely abnormal. Although most of the cases have moderate to severe airflow limitation, airflow can also be categorized. Using airflow (either a continuous or categorical variable) as the dependent variable, the genotypic frequencies were compared to determine the existence of any evidence for a dose response with respect to a particular polymorphism. General linear model (GLM) was used to test for an association of genotype with continuous FEV1. However, due to small numbers of individuals within some subclasses, dose responses for every race-gender subclass could not be detected. The gene-environment (smoking) interaction was investigated in a sub-analysis that included smoking severity and duration.

Haplotype frequencies were estimated using the expectation-maximization (EM) algorithm, as haplotypes could not be discerned directly from double heterozygotes. Random effects model (REM) was used to get an insight into the association of β₂-AR genotypes with the nature of longitudinal changes occurring over time in various outcome variables. For each subject, a random intercept and random slope was estimated from the base model. The slopes were calculated as the difference between Year 1 and Year 5 mean pulmonary function estimates divided by the average time between them. This model provided an estimate of the relationship between longitudinal changes in outcome variables and the genotypes, similar to that obtained from multiple regression. Addition of the interaction between genotype and time tested whether the slopes were constant over all levels of the genotype.

### EXAMPLE 9

### Results of the Study

The characteristics of the subjects in the study are summarized in Table 2. Further, the distribution of polymorphisms at amino acid positions 16 and 27 within the subjects with airflow limitation (cases) and those without airflow limitation (controls) are summarized in Table 3.

TABLE 2
Characteristics of the Study subjects.

| | Controls | | Cases | |
|---|---|---|---|---|
| Gender | Males | Females | Males | Females |
| | 286 (55.0%) | 234 (45.0%) | 148 (57.4%) | 110 (42.6%) |

| | | | | | | |
|---|---|---|---|---|---|---|
| Smoking | Never | Former | Current | Never | Former | Current |
| status | 90 | 138 | 292 | 42 | 73 | 143 |
| | (17.3%) | (26.5%) | (56.2%) | (16.3%) | (28.3%) | (55.4%) |

| | | |
|---|---|---|
| Age | 73.48 ± 2.77 | 73.16 ± 2.86 |
| PPFEV1 | 99.36 ± 16.31 | 62.65 ± 18.08 |
| PPFVC | 97.58 ± 14.61 | 80.91 ± 18.61 |
| PPPEF | 100.50 ± 22.02 | 57.87 ± 19.74 |
| Pack/yr | 26.73 ± 28.98 | 40.41 ± 33.40 |

TABLE 3
Genotype frequencies (%) in Controls and Cases.

| | | Frequency | | |
|---|---|---|---|---|
| Locus | Genotype | Controls | Cases | P-value |
| β₂AR-16 | A/A | 89 (17.1%) | 53 (20.5%) | 0.46 |
| | A/G | 248 (47.7%) | 114 (44.2%) | |
| | G/G | 183 (39.2%) | 91 (35.3%) | |
| β₂AR-27 | C/C | 232 (44.6%) | 131 (50.8%) | 0.0003 |
| | C/G | 237 (45.6%) | 83 (32.2%) | |
| | G/G | 51 (9.8%) | 44 (17.1%) | |

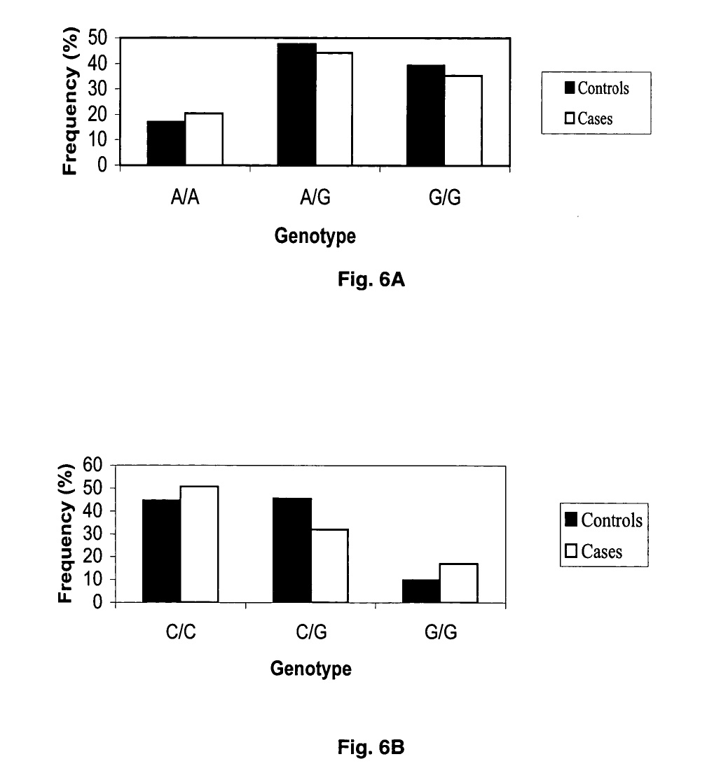

As shown in Table 3, there was significant difference in genotype frequencies between the two groups at position 27 (p=0.0003 for a 3 X 2 contingency table) but not at position 16 (P=0.46 for a 3 X 2 contingency table). Further, the frequency of homozygous Glu/Glu27 (17.1% vs 9.8%) and heterozygous Gln/Glu27 (32.2% vs 45.6%) was also significantly different between the cases and controls, respectively. The distribution of genotypes at position 16 and 27 within cases and controls is also illustrated (FIGS. 6A and 6B). It was observed that heterozygosity for the C/G genotype was significantly more frequent in controls suggesting a protective effect of this genotype while homozygosity for the G/G genotype was significantly more frequent in cases compared to controls (FIG. 6B).

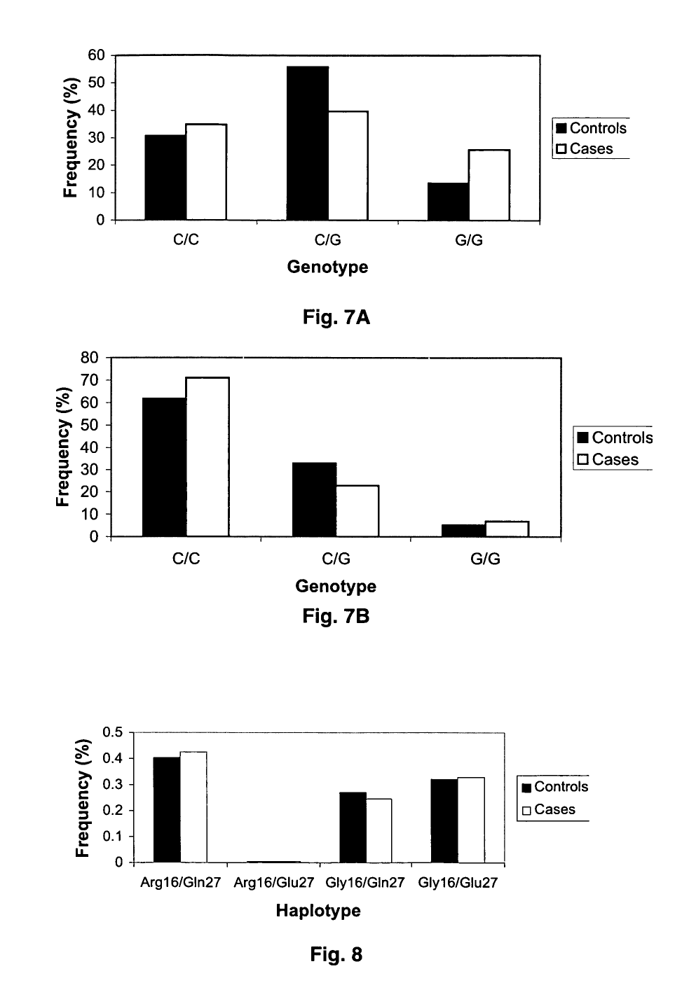

When stratified by race as shown in table 4, it was observed that although there was a significant difference in genotype frequencies between the case and controls at position 27 for Whites (P=0.001 for a 3 X 2 contingency table), there was no significant difference observed between the two groups for Blacks (P=0.15 for a 3 X 2 contingency table). Additionally, there was no significant difference observed for either the two races at position 16. It was observed that heterozygosity for the C/G genotype was significantly more frequent in controls and homozygosity for the G/G genotype was significantly more frequent in cases. The distribution of genotypes at position 27 for Whites and Blacks is also illustrated (FIGS. 7A, 7B).

TABLE 4
Genotype frequencies (%) in Controls and Cases stratified by race.

| | | | Frequency | | |
|---|---|---|---|---|---|
| Locus | Race | Genotype | Controls | Cases | P-value |
| β₂AR-16 | Whites | A/A | 41 (14.3%) | 26 (18.1%) | 0.33 |
| | | A/G | 132 (46.0%) | 56 (38.9%) | |
| | | G/G | 114 (39.7%) | 62 (43.1%) | |
| | Blacks | A/A | 48 (20.6%) | 27 (23.7%) | 0.66 |
| | | A/G | 116 (49.8%) | 58 (50.9%) | |
| | | G/G | 69 (29.6%) | 29 (25.4%) | |
| β₂AR-27 | Whites | C/C | 88 (30.7%) | 50 (34.7%) | 0.001 |
| | | C/G | 160 (55.8%) | 57 (39.6%) | |
| | | G/G | 39 (13.6%) | 37 (25.7%) | |
| | Blacks | C/C | 144 (61.8%) | 81 (71.1%) | 0.15 |
| | | C/G | 77 (33.1%) | 26 (22.8%) | |
| | | G/G | 12 (52.0%) | 7 (6.1%) | |

In addition to analyzing individual polymorphisms, haplotype frequencies were also estimated using the EM algorithm. No significant association was observed between the haplotypes and airflow limitation or pulmonary function either with or without stratification by race (FIG. 8).

The excess homozygosity for Glu/Glu27 and Gln/Gln27 in cases resulted in a deviation from Hardy-Weinberg equilibrium (P<0.0001). However, the control group was in Hardy-Weinberg equilibrium at position 27 and did not reach a statistical significance (P=0.43). Both cases and control were in Hardy-Weinberg equilibrium at position 16. However, there was no significant difference between allele frequency for polymorphisms at both positions 16 and 27 between cases and controls.

This data suggested that the heterozygous Gln/Glu27 genotype was significantly larger in controls compared to cases (45.6% vs 32.2%) and thus had a protective role in cases. To ensure that this association was not due to potentially confounding factors, a logistic regression was also performed (Table 5).

TABLE 5
Multiple logistic regressions for cases versus controls.

| Variable | Odds ratio | 95% CI | P-value |
|---|---|---|---|
| Pack year | 2.85 | 2.07 to 3.95 | <0.0001 |
| (<=40, 0; >40, 1) | | | |
| Glu/Gln27 genotype | 0.62 | 0.42 to 0.91 | 0.02 |

Additionally the FEV1 percent predicted as main dependent variable relative to genotype was also examined. Covariates included gender, race, smoking status, pack years and beta-agonist use. An association between heterozygosity for the polymorphism at position 27 and airflow limitation remained significant in this analysis. The adjusted odds ratio for the heterozygous Gln/Glu27 genotype and airflow limitation remained significant in this analysis. The adjusted odds ratio for the heterozygous Gln/Glu27 genotype and airflow limitation was 0.62 (95% CI 0.42 to 0.91, P=0.02). The only other significant covariate was pack year, with an adjusted odds ratio of 2.86 (95% CI 2.07 to 3.95, P<0.0001).

When the polymorphisms were compared with the results of the patients' FEV1, there was no significant difference at position 16 (P=0.38). However in both Whites and Blacks, there was a significant difference observed at position 27 (P=0.01) in cases. When stratified by race, it was observed that the continuous FEV1 was significantly associated with the polymorphism at position 27 in Whites (P=0.01) but was marginally significant in Blacks (P=0.09). In Whites, subjects with Glu/Glu27 genotype had a lower FEV1 (76.8; 95% CI 71.7-81.9) compared to subjects with either Gln/Gln27 (84.6; 95% CI 80.9-88.3) or Gln/Glu27 (90.0; 95% CI 87.1-92.9) genotypes.

The patients were further divided into groups based on their FEV1 values according to the proposed GOLD staging system for the severity of the airflow limitation. For example, FEV1<50% of predicted (severe); FEV1 50 to 79% of predicted (moderate); and FEV1>79% of predicted (mild). The genotypic frequencies of these polymorphisms were compared in each group (Tables 6 and 7).

TABLE 6
Genotype frequencies (%) stratified by FEV1 in Cases.

| | | FEV1% predicted | | | P- |
|---|---|---|---|---|---|
| Locus | Genotype | <50% | 50-79% | >79% | value |
| β₂AR-16 | A/A | 15 (21.4%) | 32 (22.4%) | 6 (13.3%) | 0.20 |
| | A/G | 27 (38.6%) | 60 (42.0%) | 27 (60.0%) | |
| | G/G | 28 (40.0%) | 51 (35.7%) | 12 (26.7%) | |
| β₂AR-27 | C/C | 34 (48.6%) | 76 (53.2%) | 21 (46.7%) | 0.04 |
| | C/G | 17 (24.3%) | 46 (32.3%) | 20 (44.4%) | |
| | G/G | 19 (27.1%) | 21 (14.7%) | 4 (8.9%) | |

TABLE 7
Genotype frequencies (%) stratified by FEV1 and race in Cases.

| | | | FEV1% predicted | | | |
|---|---|---|---|---|---|---|
| Locus | Race | Genotype | <50% | 50-79% | >79% | P-value |
| β₂AR-16 | Whites | A/A | 4 (11.4%) | 18 (21.0%) | 4 (21.1%) | 0.21 |
| | | A/G | 10 (28.6%) | 37 (41.1%) | 9 (47.4%) | |
| | | G/G | 21 (60.0%) | 35 (38.9%) | 6 (31.6%) | |
| | Blacks | A/A | 11 (31.4%) | 14 (26.4%) | 2 (7.7%) | 0.13 |
| | | A/G | 17 (48.6%) | 23 (43.4%) | 18 (69.2%) | |
| | | G/G | 7 (20.0%) | 16 (30.2%) | 6 (23.1%) | |
| β₂AR-27 | Whites | C/C | 7 (20.0%) | 37 (41.1%) | 6 (31.6%) | 0.02 |
| | | C/G | 12 (34.3%) | 35 (38.9%) | 10 (52.6%) | |
| | | G/G | 16 (45.7%) | 18 (20.0%) | 3 (15.8%) | |
| | Blacks | C/C | 27 (77.1%) | 39 (73.6%) | 15 (57.7%) | 0.24 |
| | | C/G | 5 (14.3%) | 11 (20.8%) | 10 (38.5%) | |
| | | G/G | 3 (8.6%) | 3 (5.7%) | 1 (3.9%) | |

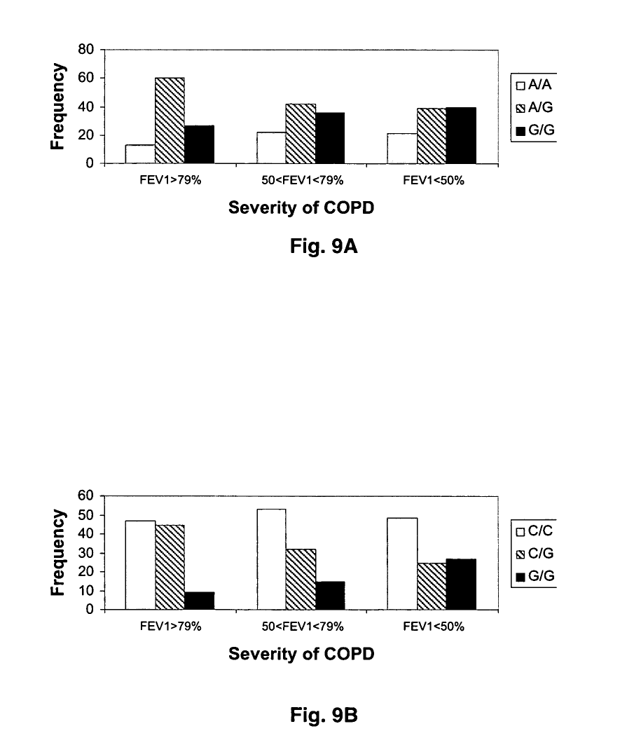

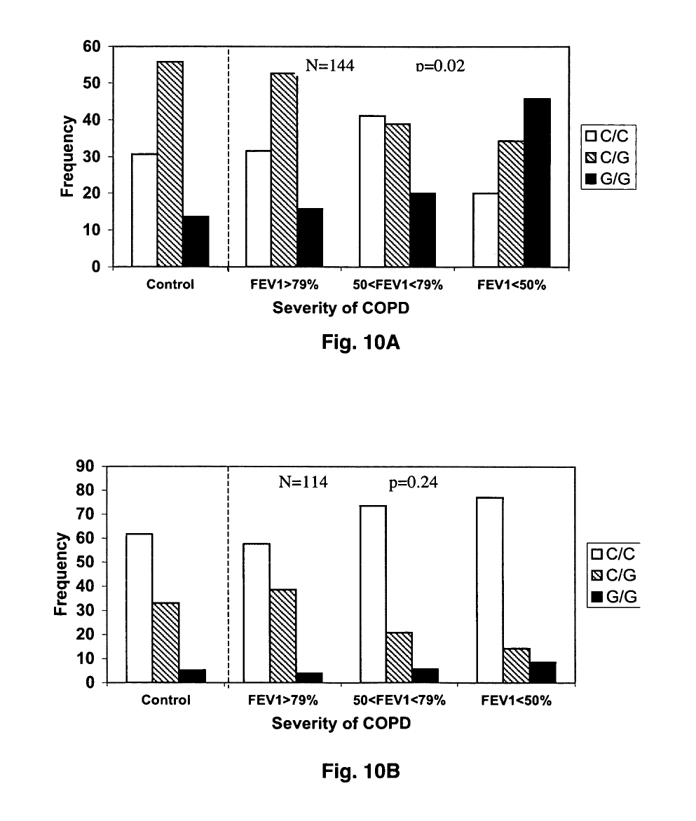

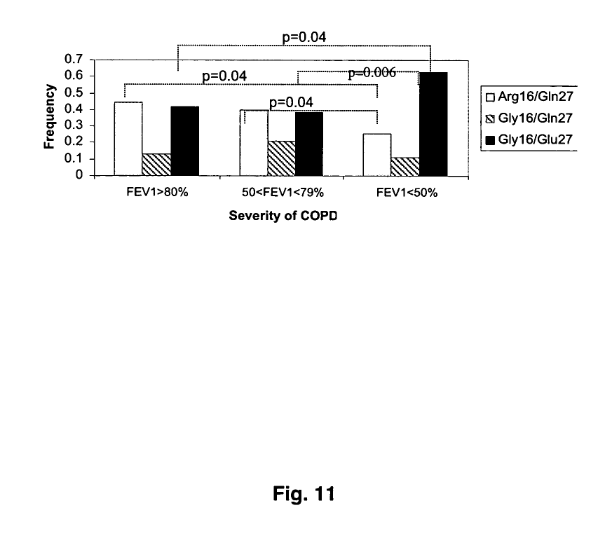

The polymorphism at position 27 showed significant correlation (P=0.04 with FEV1 percent predicted when analyzed for cases, both Blacks and Whites (FIGS. 9A and 9B). When stratified by race in cases, it was observed that FEV1 percent predicted significantly correlated with the polymorphism at position 27 only in Whites (P=0.02) but not in Blacks (P=0.24) (FIGS. 10A and 10B). Additionally, the severity of COPD was also observed to be significantly associated with the haplotype of β₂-AR at positions 16 and 27 in Whites (FIG. 11). Gly/Glu27 allele was most frequent in subjects with FEV1<50%, whereas Arg/Gln27 allele was most common in subjects with FEV1>79%.

There was no significant association observed between PPFEV6 and PPPEF data in cases and either of the two polymorphisms at position 16 and 27, respectively. PPFVC was significantly associated with the polymorphism at position 27. Subjects with homozygous Glu/Glu27 polymorphism in general, had a significantly lower PPFVC values compared to homozygous Gln/Gln27 (P=0.03) and heterozygous Gln/Glu27 (P=0.01). There was no other significant correlation between the polymorphism at position 27 and other parameters measured in the pulmonary function test.

Secondary analysis was performed for measures of functional capacity and quality of life associated with the pulmonary disease. There was a significant association between polymorphism at position 27 and the quality of life (FPHSTAT), assessed by a question regarding health in general. Subjects with heterozygous Gln/Glu27 had a better quality of life compared to homozygous Gln/Gln27 genotype and the two groups were significantly different with a P-value of 0.04. There was no significant interaction between the polymorphisms at position 16 and 27 and other measures of functional capacity like ease EASEQM, EASE1M, MTR400SD, TWOMINSD, BMI and EPESEPPB. No significant difference was observed with respect to the center of patient recruitment between the Memphis, Tenn. and Pittsburgh, Pa. vicinities. Additionally, no significant effect of gender was observed on any of the analysis.

In this study, the longitudinal results in pulmonary function measures (FEV1 and measured FEV1/FVC) after 5 years of follow-up did not show any conclusive association with β₂-AR genotypes at positions 16 and 27. However, more rapid decliners (Year 1-Year 5) in MTR400SD were observed in subjects with homozygous Glu/Glu27, whereas the rate of MTR40OSD was slowest in subjects with homozygous Gln/Gln27. Due to the physical limitation of these subjects, the 400 meters walking speed may be the best measure of decline in functional capacity in these subjects. There was also a similar trend seen in TWOMINSD, but it did not achieve the level of significance. Furthermore, EASE1M and EASEQM are both categorical variables and did not change significantly at the follow-up.

Discussion

There is a lot of controversy in the literature regarding the role of β₂-AR polymorphisms in the airway limitation (Ho, L. I. et al., 2001; Joos, L. et al., 2003; Chong, L. K. et al., 2000; Lin, Y. C. et al., 2003; Summerhill, E. et al., 2000; Leineweber, K. and Brodde, O. E., 2004). Historically, β₂-AR receptor polymorphisms have been associated with the viewpoint of asthma (Joos, L. et al., 2001; Xu, X and Weiss, S. T., 2002). Therefore, the goal of this study was to investigate the association between β₂-AR polymorphisms at positions 16 and 27 and airflow limitation, pulmonary function tests and the quality of life. In doing so, this study also demonstrates the usefulness of genotyping assay described in the present invention for detecting such β₂-AR single nucleotide polymorphisms.

The allele frequencies observed in the control group of the cohort in this study were similar to those reported previously (Xie, H. G. et al., 1999). It was observed that the distribution of genotype frequency of polymorphism at position 16 in the subjects with airflow limitation was not significantly different from that of normal subjects, whereas significant difference was observed for the polymorphism at position 27 between cases and controls. This study also demonstrated a significant association between Glu27 β₂-AR polymorphism and the severity of airflow limitation, which was represented by FEV1 percent predicted.

Additionally, the data obtained in this study suggested that heterozygous Gln/Glu27 genotype had a protective role in cases. This observation is consistent with that of Joos and coworkers, who observed that the heterozygosity at position 27 might be protective against an accelerated rate of decline in lung function. The beneficial effect of heterozygous genotype (heterozygous advantage) has been well documented in the literature in several diseases with distinct phenotypes. The possible explanation as suggested by Joos et al. could be that Gln/Glu “heterodimers” have somehow altered the functional characteristics compared with either Gln/Gln or Glu/Glu homodimers. An alternative explanation could be related to linkage disequilibrium between Gln27 polymorphism and an additional polymorphism in β₂-AR or closely related genes in near vicinity.

Further in addition to analyzing individual polymorphisms, EM algorithm was used to estimate haplotype frequencies. No significant association of the haplotypes with the airflow limitation and the pulmonary function was observed. There are many additional polymorphisms in the β₂-AR, which could be used to further define the haplotypes in the population of this study. However, it has been demonstrated by a previous study that the polymorphisms at position 16 and 27 are sufficient to distinguish between the three common haplotypes in Caucasian population (Drysdale, C. M. et al., 2000). It is therefore unlikely that additional haplotypic data would change the results of this study to a significant extent.

This study also demonstrated strong linkage disequilibrium between codon 16 and codon 27 (P<0.0001). There is evidence in the literature regarding the association of Glu27 in the homozygous state with Gly16 and that Gln27 in the homozygous state can be co-expressed either with Arg16 or Gly16 (Leineweber, K. and Brodde, O. E., 2004). Arg16/Glu27 variant was particularly rare in the sample population of this study. Additionally, in this study the haplotypes at β₂-AR positions 16 and 27 were observed to be significantly associated with the severity of airflow limitation. Gly/Glu27 allele was most frequent in subjects with severe form of COPD, whereas Arg/Gln27 allele was most common in subjects with milder form of the disease.

No conclusive association of the longitudinal decline in pulmonary function (FEV1 and FEV1/FVC) with β₂-AR genotypes at positions 16 and 27 was observed. One of the plausible reasons for this could be that the longitudinal decline in lung function was based on just two measurements at Year 1 and Year 5. As the precision of the estimation of individual slopes improves with more measurements, long-term studies with multiyear measurements of change in lung function are necessary.

There is considerable evidence in literature, which suggests that the genetic factors influence both the pulmonary function and the risk of developing airflow limitation (Wilk, J. B. et al., 2003; Joost, O. et al., 2002; Malhotra, A., et al., 2003; Sandford, A. J. et al., 2001; Wilk, J. B. et al., 2003). This study demonstrated that the distributions of β₂-AR polymorphisms are different between ethnic groups. These polymorphisms might be important in the phenotypic modulation of airflow limitation and determining its severity.

The following references were cited herein:
Allen, R. A. et al. (2001) Eur J Clin Invest 31:843-851.
Benetazzo, M. G. et al. (1999) Respir Med 93(9): 648-654.
Bottema, C. D. et al. (1993) Methods Enzymol 218:388-402.
Cheng, S. L. et al. (2004)Eur Respir J 23(6): 818-824.
Chong, L. K. et al. (2000) Pharmacogenetics 10(2): 153-162.
Dialyna, I. A. et al. (2003) Oncol Rep 10(6): 1829-1835.
Drysdale et al. (2000) Proc Natl Acad Sci USA 97(19):10483-10488.
Gray M. R. (1992) Am J Hum Genet 50: 331-346.
Gupta, M. et al. (2004) Clin Chim Acta 341: 93-100.
Harrison, D. J. et al. (1997) Hum Exp Toxicol 16(7): 356-360.
Higham, M. A. et al. (2000) Eur Respir J 15(2): 281-284.
Ho, L. I. et al. (2001) Chest 120(5): 1493-1499.
Ishii, T. et al. (2000) Eur J Clin Invest 30(6): 543-548.
Ishii, T. et al. (2000) Respir Med 94(9): 847-851.
Ishii, T. et al. (1999) Thorax 54(8): 693-696.
Joos, L. et al. (2003) Thorax 58(8): 703-707.
Joos, L. et al. (2002) Hum Mol Genet 11(5): 569-576.
Joos, L. et al. (2001) Curr Opin Pulm Med 7(2): 69-74.
Joost, O. et al. (2002) Am J Resp Crit Care Med 165(6): 795-799.
Keatings, V. M. et al., (2000) Chest 118(4): 971-975.
Laufs, J. et al., (2004) Am J Pharmacogenomics 4(1): 63-68.
Leineweber, K. and Brodde, O. E. (2004) Life Sci 74(23): 2803-2814.
Liggett, S. B. (2000) Am J Respir Crit Care Med 161(3 Pt 2): S197-201.
Liggett, S. B. (2002) N Engl J Med 346(7): 536-538.
Lin, Y. C. et al. (2003) J Asthma 40(3): 265-272.
Lucas, T. et al. (2004) Clin Chem 50: 769-772.
Malhotra et al. (2003) Am J Resp Crit Care Med 168(5): 556-561.
Minetmatsu, N. et al., (2001) Biochem Biophys Res Commun 289(1): 116-119.
Newton, C. R. et al. (1989) Nucleic Acids Res 17(7): 2503-2516.
Okimoto, et al. (1996) Biotechniques 21(1): 20-22, 24, 26.
Paschke, T et al. (2001) Toxicology 168: 259-268.
Poller, W. et al. (1990) Eur J Clin Invest 20(1): 1-7.
Poller, W. et al. (1993) Genomics 17(3): 740-743.
Rodriguez, F. et al. (2002) Anal Biochem 308(1): 120-126.
Romaino, S. M. et al. (2004) J Clin Pharm Ther 29: 47-52.
Ruse, C. E. et al. (2003) J Am Geriatr Soc 51(9): 1265-1269.
Sakao, S. et al. (2001) Am J Respir Crit Care Med 163(2): 420-422.
Sandford et al. (2001) Am J Resp Crit Care Med 163(2): 469-473.
Sandford, A. J. et al. (1998) Dis Markers 13(4): 257-260.
Schellenberg et al. (1998) Am J Respir Crit Care Med 157(3 Pt 1): 957-961.
Smith, C. A. and Harrison, D. J. (1997) Lancet 350(9078): 630-633.
Sommer, S. S. et al. (1992) Biotechniques 12(1): 82-87.
Song, P. et al. (2002) AAPS PharmiSci 4: article 29: 1-6.
Summerhill, E. et al. (2000) Am J Resp Crit Care Med 162(2 Pt 1): 599-602.
Taylor, et al. (2001)Am J Pharmacogenomics 1(3): 165-174.
Taylor, et al. (2002) Pharmacogenomics 3(2):173-184.
Teramoto, S. and Ishii, T. (2001) Chest 119(1): 315-316.
van der Pouw Kraan, T. C. et al. (2002) Genes Immun 3(7): 436-439.
Wang, Z. et al. (2001) Am J Respir Crit Care Med 163(6): 1404-1409.
Waterer, G. W. et al. (2001) J Am Geriatr Soc 49(8): 1032-1038.
Wilk et al. (2003) Am J Respir Crit Care Med 167(11): 1528-1533.
Wilk, J. B. et al., (2003) Hum Mol Genet 12(21): 2745-2751.
Xu, X. and Weiss, S. T. (2001) Am J Respir Crit Care Med 166(5): 775.
Xie, H. G. et al. (1999) Pharmacogenetics 1999; 9(4): 511-516.
Yates, C. R. et al. (2003) J Clin Pharmacol 43: 555-564.
Yim, J. J. et al. (2000) Thorax 55(2): 121-125.
Yim, J. J. et al. (2002) Lung 180(2): 119-125.
Yoshikawa, M. et al. (2000) Int J Mol Med 5(1): 49-53.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

1. A primer pair for genotyping a single nucleotide polymorphism of human β₂-adrenergic receptor (β₂₋AR) gene, comprising:
an allele specific sense primer; and
a reverse primer.

2. The primer pair of claim 1, wherein said allele specific sense primer is a wild type allele specific sense primer or a mutant allele specific sense primer, wherein said allele specific sense primer has an additional internal nucleotide mismatch two to three bases from the 3′ terminus.

3. The primer pair of claim 2, wherein said wild type allele specific sense primer has a nucleotide sequence at the 3′ end at least 85% homologous to SEQ ID No. 1 or said mutant allele specific sense primer has a nucleotide sequence at the 3′ end at least 85% homologous to SEQ ID No. 2, wherein said homology is within first 16 base pairs from the 3′ end.

4. The primer pair of claim 2, wherein said wild type allele specific sense primer has a nucleotide sequence of SEQ ID No. 1 or said mutant allele specific sense primer has a nucleotide sequence of SEQ ID No. 2.

5. The primer pair of claim 2, wherein said wild type allele specific sense primer has a nucleotide sequence at the 3′ end at least 85% homologous to SEQ ID No. 4 or said mutant allele specific sense primer has a nucleotide sequence at the 3′ end at least 85% homologous to SEQ ID No. 5, wherein said homology is within the first 16 base pairs from the 3′ end.

6. The primer pair of claim 2, wherein said wild type allele specific sense primer has a nucleotide sequence of SEQ ID No. 4 or said mutant allele specific sense primer has a nucleotide sequence of SEQ ID No. 5.

7. The primer pair of claim 1, wherein said reverse primer has a nucleotide sequence about 90% homologous to SEQ ID No.: 3 or to SEQ ID No. 6.

8. The primer pair of claim 1, wherein said reverse primer has a nucleotide sequence of SEQ ID No.: 3 or SEQ ID No. 6.

9. The primer pair of claim 1, wherein said single nucleotide polymorphism of the human β₂-adrenergic receptor (β₂₋AR) gene is an A46G or a C79G single nucleotide polymorphism.

10. A method of genotyping a single nucleotide polymorphism of human β₂-adrenergic receptor (β₂₋AR) gene, comprising the steps of:
extracting DNA from sample of an individual;
amplifying said DNA in separate PCR reactions comprising wild type allele specific primer and a reverse primer and a mutant allele specific primer and said reverse primer; and
identifying the products of said DNA amplification, wherein the presence of products amplified by said wild type allele specific sense primer and said reverse primer and said mutant allele specific sense primer and said reverse primer indicate that the individual has said single nucleotide polymorphism of human β₂-adrenergic receptor (β₂₋AR) gene.

11. The method of claim 10, further comprising:
evaluating clinical significance of said single nucleotide polymorphism of β₂-adrenergic receptor (β₂₋AR) gene in individuals with disease.

12. The method of claim 11, wherein said evaluating step comprises:
comparing the incidence of the polymorphism in said individuals to the incidence of the polymorphism in samples from control individuals who do not have the disease, wherein increased incidence of the polymorphism in individuals with said disease compared to said control individuals indicates that said polymorphism is clinically significant in said disease.

13. The method of claim 11, further comprising:
correlating the incidence of said polymorphism with said disease in the population of individuals of same ethnicity to determine prevalence, severity and response to treatment in said population.

14. The method of claim 11, wherein said disease is respiratory or cardiovascular disease, wherein said respiratory or cardiovascular disease is asthma, chronic obstructive pulmonary disease or hypertension.

15. The method of claim 10, wherein said wild type or said mutant allele specific sense primers have an additional internal nucleotide mismatch two-three bases from the 3′ terminus.

16. The method of claim 10, wherein said single nucleotide polymorphism of human β₂-adrenergic receptor (β₂₋AR) gene is an A46G or a C79G single nucleotide polymorphism.

17. The method of claim 16, wherein primers for genotyping said A46G single nucleotide polymorphism comprises a wild type allele specific sense primer having a nucleotide sequence at the 3′ end at least 85% homologous to SEQ ID No. 1, a mutant allele specific sense primer having a nucleotide sequence at the 3′ end at least 85% homologous to SEQ ID No. 2 and a reverse primer having a nucleotide sequence about 90% homologous to SEQ ID No. 3, wherein said homology at the 3′ end is within the first 16 base pairs.

18. The method of claim 16, wherein primers for genotyping said A46G single nucleotide polymorphism comprises a wild type allele specific sense primer having a nucleotide sequence of SEQ ID No. 1, a mutant allele specific sense primer having a nucleotide sequence of SEQ ID No. 2 and a reverse primer having a nucleotide sequence of SEQ ID No. 3.

19. The method of claim 16, wherein primers for genotyping said C79G single nucleotide polymorphism comprises a wild type allele specific sense primer having a nucleotide sequence at the 3′ end at least 85% homologous to SEQ ID No. 4, a mutant allele specific sense primer having a nucleotide sequence at the 3′ end at least 85% homologous to SEQ ID No. 5 and a reverse primer with a nucleotide sequence about 90% homologous to SEQ ID No. 6, wherein said homology at the 3′ end is within the first 16 base pairs.

20. The method of claim 16, wherein the primers for genotyping said C79G single nucleotide polymorphism comprises a wild type allele specific sense primer having a nucleotide sequence of SEQ ID No. 4, a mutant allele specific sense primer having a nucleotide sequence of SEQ ID No. 5 and a reverse primer having a nucleotide sequence of SEQ ID No. 6.

21. The method of claim 10, wherein the identification of a product having 204 base pairs corresponds to a product with A46G and the identification of a product having 158 base pairs corresponds to a product with C79G single nucleotide polymorphisms of human β₂-adrenergic receptor (β₂₋AR) gene.

22. A kit for genotyping a single nucleotide polymorphism of human β₂-adrenergic receptor (β₂₋AR) gene, comprising:
a wild type allele specific sense primer;
a mutant allele specific sense primer; and
a reverse primer.

23. The kit of claim 22, wherein both of said allele specific sense primers have an internal nucleotide mismatch two or three bases from the 3′ terminus.

24. The kit of claim 22, wherein said single nucleotide polymorphism of human β₂-adrenergic receptor (β₂₋AR) gene is an A46G or a C79G single nucleotide polymorphism.

25. The kit of claim 24, wherein primers for genotyping said A46G single nucleotide polymorphism comprises a wild type allele specific sense primer having nucleotide sequence at the 3′ end at least 85% homologous to SEQ ID No. 1, a mutant allele specific sense primer having nucleotide sequence at the 3′ end at least 85% homologous to SEQ ID No. 2 and a reverse primer having nucleotide sequence about 90% homologous to SEQ ID No. 3, wherein the homology at the 3′ end is within first 16 base pairs.

26. The kit of claim 24, wherein the primers for genotyping said A46G single nucleotide polymorphism comprises a wild type allele specific sense primer having a nucleotide sequence of SEQ ID No. 1, a mutant allele specific sense primer having a nucleotide sequence of SEQ ID No. 2 and a reverse primer having a nucleotide sequence of SEQ ID No. 3.

27. The kit of claim 24, wherein the primers for genotyping said C79G single nucleotide polymorphism comprises a wild type allele specific sense primer having a nucleotide sequence at the 3′ end at least 85% homologous to SEQ ID No. 4, a mutant allele specific sense primer having a nucleotide sequence at the 3′ end at least 85% homologous to SEQ ID No.5 and a reverse primer having a nucleotide sequence about 90% homologous to SEQ ID No. 6, wherein said homology at the 3′ end is within first 16 base pairs.

28. The kit of claim 24, wherein the primers for genotyping said C79G single nucleotide polymorphism comprises a wild type allele specific sense primer having a nucleotide sequence of SEQ ID No. 4, a mutant allele specific sense primer having a nucleotide sequence of SEQ ID No. 5 and a reverse primer having a nucleotide sequence of SEQ ID No. 6.


SEQUENCE LISTING (8 sequences)

SEQ ID NO: 1 - DNA, 20 bp, artificial sequence
  46FW primer for B2-AR at pos 1614-1633
cttcttgctggcacccatta

SEQ ID NO: 2 - DNA, 20 bp, artificial sequence
  46FM primer for B2-AR at pos 1614-1633
cttcttgctggcacccattg

SEQ ID NO: 3 - DNA, 20 bp, artificial sequence
  46R primer for B2-AR at pos 1817-1798
caggccagtgaagtgatgaa

SEQ ID NO: 4 - DNA, 20 bp, artificial sequence
  79FW primer for B2-AR at pos 1647-1666
ggaccacgacgtcacgcaac

SEQ ID NO: 5 - DNA, 20 bp, artificial sequence
  79FM primer for B2-AR at pos 1647-1666
ggaccacgacgtcacgcaag

SEQ ID NO: 6 - DNA, 20 bp, artificial sequence
  79R primer for B2-AR at pos 1804-1785
tgatgaagtagttggtgacc

SEQ ID NO: 7 - DNA, 18 bp, artificial sequence
  sense primer for B2-AR
gctcacctgccagactgc

SEQ ID NO: 8 - DNA, 20 bp, artificial sequence
  antisense primer for B2-AR
cagcaggtctcattggcata